(19) United States
(12) Patent Application Publication
(10) Pub. No.: US 2026/0069857 A1
(43) Pub. Date: Mar. 12, 2026

(54) MOTOR HOUSING MODULE FOR A HEART SUPPORT SYSTEM, AND HEART SUPPORT SYSTEM AND METHOD FOR MOUNTING A HEART SUPPORT SYSTEM

(71) Applicant: KARDION GMBH, Stuttgart (DE)

(72) Inventors: Julian Kassel, Böblingen (DE); David Minzenmay, Stuttgart (DE); Thomas Alexander Schlebusch, Renningen (DE)

(21) Appl. No.: 19/296,716

(22) Filed: Aug. 11, 2025

Related U.S. Application Data
(63) Continuation of application No. 17/057,243, now Pat. No. 12,383,727.

(30) Foreign Application Priority Data
May 30, 2018 (DE) 102018208539.0

(51) Int. Cl. A61M 60/878; A61M 60/816

(52) U.S. Cl. CPC A61M 60/878; A61M 60/816; A61M 2205/3334; A61M 2205/8262

(57) ABSTRACT

The invention relates to a motor housing module (110) for sealing a motor compartment of a motor of a heart support system. The motor housing module (110) has at least one feed-through portion (205), at least one feed-through line (210), and at least one contact pin (215). The feed-through portion (205) is designed to establish an electrical connection between the heart support system and a connection cable in order to externally contact the heart support system. The at least one feed-through line (210) is embedded in the feed-through portion (205) and extends through the feed-through portion (205). The feed-through line (210) can be connected to the motor and to the connection cable. A first end of the at least one contact pin (215) is embedded in the feed-through portion (205) and a second end of the contact pin (215) projects from the feed-through portion (205) on a side facing away from the motor compartment. The second end of the contact pin (215) can be connected to a sensor line to at least one sensor of the heart support system and to the connection cable.

### INCORPORATION BY REFERENCE TO ANY PRIORITY APPLICATIONS

The present application is a continuation of U.S. application Ser. No. 17/057243 filed on May 30, 2019, which is a National State entry of PCT Patent Application No. PCT/EP2019/064156, filed on May 30, 2019, titled MOTOR HOUSING MODULE FOR A HEART SUPPORT SYSTEM, AND HEART SUPPORT SYSTEM AND METHOD FOR MOUNTING A HEART SUPPORT SYSTEM, which is an International Application of and claims the benefit of priority to German Patent Application No. 102018208539.0, filed on May 30, 2018, the contents of each of which are hereby incorporated by reference herein in their entirety as if fully set forth herein for all purposes. Any and all applications for which a foreign or domestic priority claim is identified in the Application Data Sheet as filed with the present application are hereby incorporated by reference under 37 CFR 1.57.

### BACKGROUND

### Field

The invention relates to a motor housing module for sealing a motor compartment of a motor of a heart support system and to a heart support system and a method for mounting a heart support system.

### Description of the Related Art

Heart support systems, such as a left ventricular heart support system, can be implanted into a heart chamber and have integrated electronic components, such as sensors. Electronic components are mostly integrated into the heart support system in the traditional manner, constructed on substrates, e.g., circuit boards or printed circuit boards (PCBs), and integrated into correspondingly sized cavities of the heart support system. These heart support systems can be implanted by means of a sternotomy, for example. In addition, it is possible to implant more compactly constructed heart support systems, for example also left ventricular heart support systems, into a blood vessel in a minimally invasive manner. Due to the installation size requirements, these more compactly constructed heart support systems do not yet have any integrated electronic components with implanted processing electronics.

U.S. Pat. No. 9,474,840 B2 describes the integration of an optical pressure sensor into the tip of a more compactly constructed heart support system for minimally invasive implantation. The optical supply line is elaborately realized by means of a glass fiber in a channel. The entire evaluation electronics are positioned remotely in an extracorporeal control console as a result of the glass fiber.

US For fully implanted systems, however, it is also necessary to implant the processing electronics.

### SUMMARY

The object of the invention is to provide an improved heart support system. It is in particular an object of the invention to create electrical connection possibilities in a heart support system in a small installation space both for a motor for driving a blood pump supporting the heart function and for sensors.

This object is achieved by a motor housing module having the features specified in Claim 1 and by a heart support system according to Claim 14 and the method specified in Claim 15 for mounting a heart support system.

Advantageous embodiments of the invention are specified in the dependent claims.

In light of this background, the approach presented here presents a motor housing module for sealing a motor compartment of a motor of a heart support system, a heart support system, and a method for mounting a heart support system according to the main claims. Advantageous developments and improvements of the device specified in the independent claim are possible by means of the measures listed in the dependent claims.

This approach presents a motor housing module for a heart support system. The motor housing module can seal the motor compartment of the heart support system in a fluid-tight manner and connect the motor of the heart support system to a connection cable via which the motor can be supplied with power. In addition, by means of the motor housing module, sensor signals can be combined, processed, and forwarded via the connection cable. The motor housing module and the heart support system can advantageously be designed to be so compact that they can be used, for example, for a left ventricular heart support system (LVAD, left ventricular assist device) for minimally invasive implantation as a fully implanted system. The heart support system can in particular be designed such that it can be inserted into a ventricle or the aorta by means of a catheter.

It is thus advantageously possible to integrate electronic components even in a compactly constructed heart support system.

A motor housing module for sealing a motor compartment of a motor of a heart support system is presented. The motor housing module has a feed-through portion, at least one feed-through line, and at least one contact pin. The feed-through portion is designed to establish an electrical connection between the heart support system and a connection cable for externally contacting the heart support system. The at least one feed-through line is embedded in the feed-through portion and extends through the feed-through portion. The feed-through line can be connected to the motor and the connection cable. A first end of the at least one contact pin is embedded in the feed-through portion, and a second end projects from the feed-through portion on a side facing away from the motor compartment. The second end of the contact pin can be connected to a sensor line to at least one sensor of the heart support system and to the connection cable.

The motor housing module can be designed in one or two parts, for example. For example, the motor housing module can have titanium components or glass components. The heart support system can, for example, be a left ventricular heart support system that has a heart pump with a motor. The motor compartment can, for example, be a portion of the heart support system, e.g., also a housing portion. The motor compartment can advantageously be sealed hermetically, i.e., in a fluid-tight manner, by means of the housing presented here. The motor housing module can, for example, consist of a material that allows a weld connection between the motor or the motor compartment and the motor housing module in order to seal the motor compartment. The feed-through portion for establishing an electrical connection between the heart support system and the connection cable can be designed in one part, for example. Alternatively, the feed-through portion can, for example, comprise a milled part and a glass component which are hermetically connected to one another by laser welding or sintering, for example. The feed-through line and the contact pin can, for example, consist of an electrically conductive material, e.g., a metal, such as an iron-nickel-cobalt alloy, with a low heat expansion coefficient or stainless steel. The connection cable for externally contacting the heart support system can, for example, establish an electrical connection to another implanted component, e.g., a power source and/or control unit of the heart support system. The sensor line can, for example, comprise a group of lines and be designed to forward sensor signals of a sensor in the pump head of the heart support system and/or sensor signals of several sensors. The sensor line can, for example, be realized as an applied flexible thin-film substrate.

According to one embodiment, the feed-through portion can have at least one through-opening filled with an electrically insulating material for embedding the at least one feed-through line and at least one blind hole filled with an electrically insulating material for embedding the at least one contact pin. The feed-through portion can thus advantageously be produced of glass, for example, and both the feed-through line and the contact pin can be embedded. This embodiment advantageously allows a particularly cost-saving production.

It is also advantageous according to one embodiment if the at least one feed-through line and, additionally or alternatively, the at least one contact pin are cylindrical or cup-shaped. If the at least one feed-through line and the at least one contact pin are designed to be cylindrical, i.e., as straight pins, the connection cable can be connected, for example, by soldering, gluing, crimping, or welding the connection cable strands directly to the pin or by using a sleeve or a plug. In the case of a cup-shaped or tulip-shaped forming of the at least one feed-through line and, additionally or alternatively, of the at least one contact pin, the connection to the connection cable can occur, for example, by inserting the strands of the connection cable into the cup of the through line or of the contact pin, wherein the fixing can be realized by means of soldering, gluing, crimping, or welding. According to this embodiment, various application forms can advantageously be realized, which is advantageous with respect to the simplest possible design. In addition, an additional mechanical stabilization of the connection can occur by means of a plug as part of the connection, for example.

According to one embodiment, the motor housing module can comprise a body. The body can have a sensor groove for accommodating at least one electronic component, in particular a sensor, and additionally or alternatively a sensor hub. A sensor can thus advantageously be positioned on the body of the motor housing module, which enables a compact design. The electrical contacting of an electronic component accommodated in the sensor groove with the feed-through portion can, for example, take place by means of an electrically conductive substrate, e.g., a flexible thin-film substrate. The sensor groove can also be formed as a depression or as a cavity, for example. The body can be a milled part made of titanium, for example. The body can be formed, for example, in order to enclose the feed-through portion. The feed-through portion, which can, for example, have glass, can then be hermetically joined to the milled part by laser welding, sintering, or injection molding. Integration of the feed-through portion into the body can be advantageous with respect to the design since the body of the motor housing module can be welded particularly easily to another portion of the heart support system, e.g., the motor compartment or the motor.

If the motor housing module according to one embodiment has a sensor groove, the motor housing module can additionally have a sensor cap for covering the at least one electronic component accommodated in the sensor groove. The sensor cap can, for example, have a metal and be fixed by gluing. This advantageously allows an accommodated electronic component to be protected by the sensor cap.

In addition, according to this embodiment, the motor housing can have a sensor line portion of the sensor line. In the region of the sensor groove, the sensor line portion can form a sensor carrier for connecting the at least one electronic component. The sensor line portion represents a part of the sensor line of the heart support system; the sensor line can be designed modularly for this purpose, for example. For forming the sensor carrier, the sensor line can expand in the region of the sensor line portion, for example. Advantageously, according to this embodiment, connection to the sensor line and integration of an electronic component, such as an additional sensor, is possible in a particularly space-saving and simple manner.

According to one embodiment, the electronic component can have a sensor hub. The sensor hub can be designed to process at least one sensor signal of the at least one sensor of the heart support system. Additionally or alternatively, the sensor hub can be designed to provide the sensor signal via the at least one contact pin to the connection cable. For example, the sensor hub can be understood to be a device that connects nodes of several sensors to one another in the shape of a star, for example. The sensor hub may be a computer network. The sensor hub may be referred to as a coupling element of several sensors. The sensor hub can, for example, connect the sensor at the pump head to a sensor accommodated in the sensor groove of the motor housing module. The connection of several sensors by means of a sensor hub can be advantageous in order to increase reliability with respect to a physical bus network. The sensor hub can, for example, comprise calibration and identification information of the pump and of the sensors of the heart support system and can be read via a communication bus in the connection cable by a central control device of the heart support system. In this way, the control device can be parameterized with motor data, for example. The sensor hub can also be used to pre-process, e.g., to aggregate, to filter, or to calibrate, sensor data of the sensors of the pump and to translate the communication protocol of the sensors into a more robust communication protocol and add artificial redundancy or checksums.

According to one embodiment, the sensor line portion can advantageously have a contact portion. The contact portion can be arranged on a side of the feed-through portion facing away from the motor compartment. In addition, the contact portion can be O-shaped or U-shaped. The contact portion can advantageously be used for electrically contacting the sensor line with the feed-through portion, wherein this embodiment is particularly space-saving. For this purpose, the contact portion can be formed, for example, as an end portion of the sensor line portion and can be folded on or onto the feed-through portion, wherein as a result of the O-shape or U-shape, the contacting of the connection cable with the at least one contact pin can, for example, be realized without contact of the contact portion to the feed-through line.

According to one embodiment, the contact portion can have at least one contact surface for connection to the at least one contact pin. The contact surface can be formed in order to at least partially enclose the at least one contact pin. For this purpose, the contact surface can be semicircular or elliptical, for example. The contact surface can, for example, have an exposed electrically contactable area, wherein the electrical contact between the sensor line portion and the contact pin can be established by solder or adhesive, for example.

According to one embodiment, the motor housing module can have a connection point cap for covering a connection point between the feed-through portion and the connection cable. This is advantageous in order to protect the connection point. The connection point cap can also be a part of the sensor cap, for example. The connection point cap, like the sensor cap, can be filled with a casting compound, e.g., a silicone or epoxy resin, in order to protect sensors and contact points from corrosion and conductive liquids. The connection point cap can be flexibly formed in order to be able to realize bend protection and strain relief in addition to mechanical protection.

In addition, according to one embodiment, the motor housing module can have a coupling device for coupling an insertion device for inserting the heart support system to the motor housing module, wherein the coupling device can in particular have at least one fixing element. This is advantageous in order to be able to, for example, fix the motor housing module in a form-fitting and/or force-fitted manner to the insertion device in order to, for example, be able to introduce the heart support system, which comprises the motor housing module, in a minimally invasive manner and to decouple it after successful implantation of the insertion device in order to release the heart support system at the destination. The fixing element can, for example, have a clamp or the like. According to one embodiment, the coupling device can be realized on the body of the motor housing module.

A heart support system is also presented. The heart support system has a housing with a motor compartment, a motor arranged in the motor compartment, at least one sensor, a sensor line electrically connected to the at least one sensor, a connection cable for externally contacting the heart support system, and an embodiment of the aforementioned motor housing module as part of the housing. The motor and the at least one sensor are electrically connected to the connection cable by means of the motor housing module.

The heart support system can be a ventricular heart support system, in particular a left ventricular heart support system. The heart support system can, for example, have an electric motor or an electrically operated motor-clutch-pump unit. The sensor can be arranged, for example, on the pump head and, additionally or alternatively, on the motor housing module. The sensor can, for example, be a pressure sensor or a sensor for measuring the blood flow direction. The heart support system can, for example, be cylindrical for minimally invasive insertion and have a diameter that is smaller than that of the human aorta, e.g., 5 to 12 millimeters.

In addition, a method for mounting a heart support system is presented. The heart support system has a motor, a motor compartment, at least one sensor, a sensor line electrically connected to the at least one sensor, and a connection cable for externally contacting the heart support system. The method comprises a step of providing, a step of establishing, a step of contacting, and a step of producing. In the step of providing, an embodiment of the aforementioned motor housing module is provided. In the step of establishing, an electrically conductive connection is established between the at least one feed-through line of the motor housing module and the motor of the heart support system. In the step of producing, a firmly bonded connection is produced between the motor housing module and the heart support system in order to seal the motor compartment of the heart support system. In the step of contacting, the at least one contact pin of the motor housing module is contacted with the sensor line of the heart support system.

The firmly bonded connection can be produced by welding, for example. Optionally, after welding, a sensor cap and, additionally or alternatively, a connection point cap for covering and protecting an electronic component or an electrically conductive interface of a component of the heart support system can also be mounted.

According to one embodiment, the method can also comprise a step of connecting the connection cable of the heart support system to the at least one feed-through line and the at least one contact pin of the motor housing module. The step of connecting can take place before or after the step of producing. If the step of connecting is after the step of producing, the motor housing module can have a passage opening for the connection cable.

This method can, for example, be implemented in software or hardware or in a mixed form of software and hardware in a control device, for example.

A computer program product or computer program having program code which can be stored in a machine-readable carrier or storage medium, such as a semiconductor memory, a hard drive memory, or an optical memory, and is used to carry out, implement, and/or control the steps of the method according to one of the embodiments described above is also advantageous, in particular if the program product or program is executed on a computer or a device.

### BRIEF DESCRIPTION OF THE DRAWINGS

Advantageous exemplary embodiments of the approach presented here are shown in the drawings and explained in more detail in the following description. The drawings show:

FIG. 1 a schematic illustration of a heart support system according to an exemplary embodiment;

FIG. 2 a schematic illustration of a motor housing module according to an exemplary;

FIG. 3 a schematic illustration of a motor housing module according to an exemplary embodiment;

FIG. 4 a schematic illustration of a motor housing module according to an exemplary embodiment;

FIG. 5 a schematic illustration of a sensor line portion of a motor housing module according to an exemplary embodiment;

FIG. 6 a schematic illustration of a cap element for a motor housing module according to an exemplary embodiment;

FIG. 7 a schematic illustration of a motor housing module according to an exemplary embodiment;

FIG. 8 a schematic illustration of a motor housing module according to an exemplary embodiment; and

FIG. 9 a flow diagram of a method for mounting a heart support system according to an exemplary embodiment.

### DETAILED DESCRIPTION

In the following description of favorable exemplary embodiments of the present invention, the same or similar reference signs are used for the elements which are shown in the various figures and have a similar effect, wherein a repeated description of these elements is omitted.

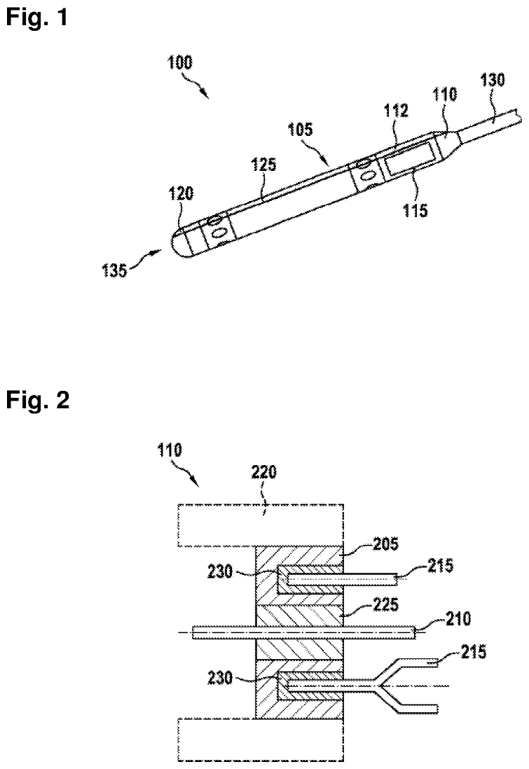

FIG. 1 shows a schematic illustration of a heart support system 100 according to an exemplary embodiment. Shown is a side view of the heart support system 100, which is designed here, by way of example, as a left ventricular heart support system 100. The heart support system 100 has a housing 105. As part of the housing 105, the heart support system 100 comprises a motor housing module 110. A motor compartment 112 is enclosed by the housing 105 and the motor housing module 110. A motor 115 is arranged in the motor compartment 112. At least one sensor 120 is arranged in a sensor assembly on a head side of the heart support system 100. The sensor 120 is electrically connected to a sensor line 125. The sensor line 125 is laid here, by way of example, across the housing 105 to the motor housing module 110; it can also run at least in portions within the housing 105 or be laid in the shape of a spiral across the housing 105. The sensor 120 can, for example, be a pressure sensor or a flow sensor for blood flow measurement by means of ultrasound or laser, for example. On the side of the motor housing module 110 facing away from the motor compartment 112, the heart support system 100 has a connection cable 130 for externally contacting the heart support system 100. The motor housing module 110 can be referred to as an electrical connecting element: The motor 115 and the at least one sensor 120 are electrically connected to the connection cable 130 by means of the motor housing module 110. The motor housing module 110, also called the motor backend, is formed to hermetically seal the motor compartment 112 and thus to seal it in a fluid-tight manner. In addition, the motor housing module 110 is designed to establish an electrical connection between the hermetically sealed motor interior of the motor 115 and the surroundings of the heart support system 100: The motor housing module 110 assumes the tasks of joining the sensor line 125, which conducts electrical signals from a pump head 135 of the heart support system 100 to the motor housing module 110, to the connection cable 130, which forwards the sensor signals and supplies the motor with electrical energy. For this purpose, electrical conductors from the interior of the motor 115 can be joined to the sensor cable 125 laid on the outside of the motor 115 and to the connection cable 130, also called supply cable. In this way, a mechanically secure connection of the connection cable 130 to the motor housing module 110 can be established. Via the connection cable 130, the heart support system 100 can be connected to another component, such as an energy source, a data processing device, or a control device.

The heart support system 100 has a cylindrical, elongated structure with a substantially constant outer diameter and rounded, tapered ends for easy positioning by means of a catheter in a blood vessel, e.g., the aorta. The motor housing module 110 has the shape of a truncated cone. It is conically formed, with a base surface in the direction of the motor compartment 112, which corresponds to the outer diameter of the heart support system 100, and with a smaller top surface as a transition to the connection cable 130.

FIG. 2 shows a schematic illustration of a motor housing module 110 for sealing a motor compartment of a motor of a heart support system according to an exemplary embodiment. The motor housing module 110 corresponds or is similar to the motor housing module 110 of FIG. 1. A cross-section of a side view of the motor housing module 110 is shown. The motor housing module 110 has at least one feed-through portion 205 for establishing an electrical connection between the heart support system and a connection cable for externally contacting the heart support system. In addition, the motor housing module 110 has at least one feed-through line 210, which is embedded in the feed-through portion 205 and extends through the feed-through portion 205. The feed-through line 210 can be connected to the motor and the connection cable of the heart support system. The motor housing module 110 furthermore has at least one contact pin 215. For example, two differently formed contact pins 215 are shown here. A first end of the contact pin 215 is embedded in the feed-through portion 205 and a second end projects from the feed-through portion 205 on a side facing away from the motor compartment. The second end of the contact pin 215 can be connected to a sensor line to at least one sensor of the heart support system and to the connection cable.

As in the exemplary embodiment shown here, the feed-through portion 205 can have at least one through-opening 225 filled with an electrically insulating material for embedding the at least one feed-through line 210 and at least one blind hole 230 filled with an electrically insulating material for embedding the at least one contact pin 215. One of the blind holes can also be filled conductively, e.g., with an electrically conductive adhesive, in order to establish an electrical connection between the motor housing and a conductor of the connection cable. This can serve to electrically shield the motor and connection cable, for example. The feed-through portion 205 is formed from titanium, for example. The through-opening 225 and the two blind holes 230 shown are formed in the feed-through portion 205 and filled, for example, with glass as electrically insulating material. The blind holes 230 can accordingly also be referred to as blind glass feed-throughs since they do not lead all the way into the interior of the hermetically sealed motor. The feed-through line 210, which can be realized as a feed-through pin or pin, is used to electrically contact the motor. The contact pins 215, also called blind pins, are used to rewire the sensor line. The feed-through line 210 as well as the at least one contact pin 215 are formed from an electrically conductive material, e.g., from a metal, such as an iron-nickel-cobalt alloy, with a low thermal expansion coefficient or such as stainless steel.

The at least one feed-through line 210 and/or the at least one contact pin 215 can be designed to be cylindrical, i.e., as straight pins, as shown here by way of example in the case of the feed-through line 210 and the upper of the two contact pins 215. The feed-through line 210 and/or the at least one contact pin 215 can alternatively also be cup-shaped, as shown by way of example in the case of the lower of the two contact pins 215. If the feed-through line 210 and/or the at least one contact pin 215 are cylindrical, the connection cable can be connected, for example, by soldering, gluing, crimping, or welding the connection cable strands directly to the feed-through line 210 and/or the contact pin 215 or by using a sleeve or a plug. If the feed-through line 210 and/or the at least one contact pin 215 are cup-shaped, the cable connection to the connection cable can be realized by inserting the strands into the cup. Fixing can take place by soldering, gluing, crimping, or welding.

According to the exemplary embodiment shown in FIG. 2, the motor housing module 110 is designed in two parts, with a body 220 and the feed-through portion 205, which is formed, for example, as a so-called glass feed-through component. The two-part design of the motor housing module 110 is advantageous in terms of production technology. In this case, the electrical contacting of the motor and the sensor line with the feed-through portion 205 can be made possible in the interior of the motor housing module 110, hereinafter also referred to as backend, wherein motor strands can be soldered to the feed-through portion 205, for example. An advantage of the two-part design of the motor housing module 110 is that a standard glass feed-through can be used for the feed-through portion 205, which can then, for example, be hermetically joined to the body 220 designed as a milled part by laser welding, sintering, or insert molding, wherein the body can have additional features, such as the integration of clamps as fixing element of the coupling device and a sensor depression in the form of the sensor groove, as described, for example, with reference to FIG. 8. The two-part design of the motor housing module 110 is also advantageous with respect to the assembly since the following production procedure can be implemented, for example: contacting the feed-through portion 205 with the motor interior; connecting the feed-through portion 205 to the body 220 by sliding the body 220 onto and over the feed-through portion 205, for example; welding the body 220 to the motor housing 112; welding the body 220 to the feed-through portion 205; establishing an electrical connection of the sensor cable to the contact pins 215; and contacting the connection cable with the feed-through line 210 and the contact pins 215. The mounting of a cap element as a protective cap as shown in FIG. 6 can then optionally take place by casting.

The two-part design of the motor housing module 110 can be realized by a combination of a milled part as a body 220 for producing the corresponding geometry with advantageous mechanical robustness and strength and by a feed-through portion 205 with classic glass feed-throughs. The body 220 as a milled part can advantageously be formed from titanium in order to be able to weld the motor housing module 110 particularly easily and efficiently to a motor housing 112 of the motor 115, which can also consist of titanium, for example. In this way, a hermetically sealed connection can be established between the body 220 and the motor housing 112 in order to seal the motor compartment in a fluid-tight manner. The forming of the contact pins 215 as glass blind pins, i.e., as blindly ending glass feed-through, allows robust rewiring of the flexible sensor line to the connection cable on the basis of glass feed-through technology by means of the possibility of connecting the contact pins 215 to the sensor line and to the connection cable. FIG. 2 thus shows a backend or motor housing module 110 with blind pins for rewiring in the form of the two contact pins 215 shown, by way of example, in the blind holes 230.

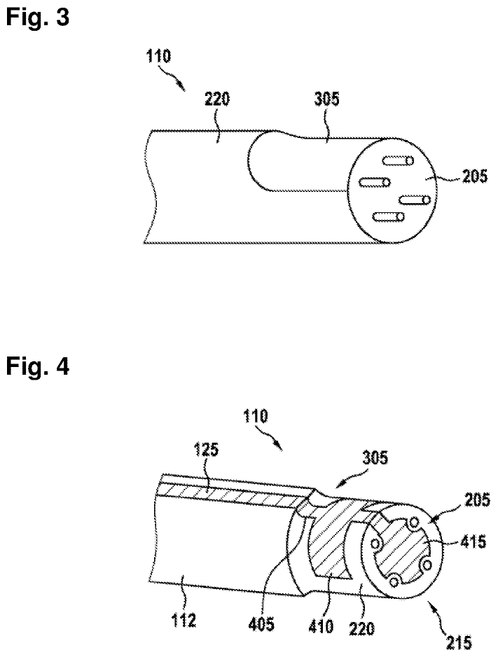

FIG. 3 shows a schematic illustration of a motor housing module 110 according to an exemplary embodiment. A side view of the motor housing module 110 with the body 220 and the feed-through portion 205 is shown, wherein the feed-through portion 205 for embedding the feed-through line and the at least one contact pin is formed and has, by way of example, recesses for this purpose.

The motor housing module 110, also called the pump backend, has a cylindrical shape with a depressed plane in the direction of the feed-through portion 205. For example, a sensor can be positioned on this depressed plane. The depressed plane can be formed as a depression or as a cavity or as a groove. According to the exemplary embodiment shown here, the body 220 correspondingly has a sensor groove 305 in the form of the depressed plane for accommodating at least one electronic component, in particular a sensor and/or a sensor hub.

An electrically conductive substrate can be arranged in the sensor groove 305 in order to realize an electrical contact of an electronic component accommodated in the sensor groove 305. The substrate can be formed, for example, in order to connect the electrical component accommodated in the sensor groove 305 to electrically conductive pins of the backend, i.e., to the at least one contact pin embedded in the feed-through portion 205. The substrate is a flexible thin-film substrate, for example. According to the exemplary embodiment shown in the following FIG. 4, the substrate can also be part of the sensor line or of a sensor line portion.

The motor housing module 110 can optionally have a coupling device for coupling an insertion device with the heart support system, as shown in FIG. 8. In addition, the motor housing module 110 can optionally have a fit for attaching a cap element as shown in FIG. 6 as a protective cap or as a bend protection grommet. The cap element can be formed, for example, in order to cover the sensor groove 305 and the feed-through portion 205.

The body 220 can be formed from the same material as the motor of the heart support system in order to be able to establish a hermetic welded connection between the motor and the backend in the form of the motor housing module 110. A fixed connection, e.g., by ultrasonic welding or injection molding of a polymer, is also possible, as well as sintering processes and glazing processes of ceramic components if the motor housing module 110 has ceramic components, for example. The feed-through portion 205, which can realize both an electrical feed-through into the hermetically sealed interior and a rewiring for the sensor line, is significant for the use of the motor housing module 110 as an electrical connecting element. Manufacturing the motor housing module 110 of one part dispenses with a weld seam and requires correspondingly formed glass feed-throughs for the feed-through portion 205.

FIG. 4 shows a schematic illustration of a motor housing module 110 according to an exemplary embodiment. A side view of the motor housing module 110 connected to the motor compartment 112 of the heart support system is shown, wherein only a proximal portion of the cylindrical heart support system comprising the motor compartment 112 is shown of the heart support system. On the side facing the motor compartment 112, the motor housing module 110 has the same diameter and the same material as the motor compartment 112. In order to form a sensor groove, the motor housing module 110 can taper conically in order to create installation space for positioning sensors. The sensor line 125 is laid here, by way of example, along the longitudinal axis of the heart support system 100 in a band-shaped manner on the housing of the heart support system across the motor chamber 112 to the motor housing module 110.

According to the exemplary embodiment shown here, the motor housing module 110 comprises a sensor line portion 405 of the sensor line 125. In the region of the sensor groove 305, the sensor line portion 405 has a sensor carrier 410 for connecting the at least one electronic component. The sensor carrier 410 can also be understood as a portion, e.g., a planar region, of the motor housing module 110. The sensor line portion 405 is formed, for example, for integrating a sensor in the sensor groove 305.

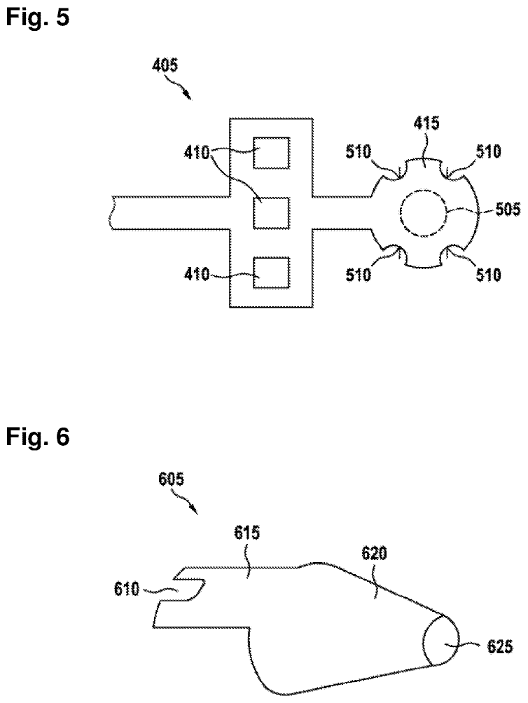

The sensor line 125 and the sensor line portion 405 can be formed from an electrically conductive flexible thin-film substrate. The sensor groove 305 is formed here in a band-shaped manner circumferentially around the motor housing module 110. The sensor line portion 405 is connected to the sensor line 125 and extends in one part along the sensor groove 305 around a portion of the lateral surface of the motor housing module 110, wherein the sensor line portion 405 is expanded in this region in order to allow several sensor carriers 410 for connecting several electronic components to be formed on the sensor line portion 405 along the sensor groove 305, as shown in the following FIG. 5. The shaping of the sensor groove 305 can be designed according to the exemplary embodiment shown here in order to enable both the cable routing of the sensor line 125 in the described portion of the sensor line portion 405 and the sensor integration on the sensor line portion 405 in the sensor groove 305. In another part, the sensor line portion 405 extends in the direction of the feed-through portion 205 from the sensor groove 305 toward the cross-sectional area of the feed-through portion 205.

According to the exemplary embodiment shown here, the sensor line portion 405 has a contact portion 415. The contact portion 415 is arranged on a side of the feed-through portion 205 facing away from the motor compartment 112. The contact portion 415 is arranged at least partially on the feed-through portion 205. The contact portion 415 can be O-shaped or U-shaped. Here, the contact portion 415 extends, by way of example, over a large part of the cross-sectional area of the feed-through portion 205. According to the exemplary embodiment shown here, the contact portion 415 has recesses in the region of the through-opening and/or of the blind holes. In order to contact the at least one contact pin 215 with the sensor line 125, the sensor line portion 405 can have an exposed, electrically contactable region in the form of an electrically conductive contact surface 510, which connects to the at least one contact pin 215 embedded in the feed-through region 205. By way of example, four contact pins 215 are shown here. The contact portion 415 has, per contact pin 215, a semicircular recess 510 adjacent to the contact pins. The sensor line portion 405, and thus the sensor line 125, is electrically connected via the contact portion 415 to the contact pins 215 in the feed-through portion 205 of the motor module housing 110. This design of the connection can also be referred to as a connection of the flexible sensor line 125, also called sensor flex, to the blind pins in the form of the contact pins 215.

FIG. 5 shows a schematic illustration of a sensor line portion 405 of a motor housing module according to an exemplary embodiment. Here, the sensor line portion 405 is designed, by way of example, as a thin-film substrate for contacting the blind pins in the form of contact pins and for integrating additional sensors of the heart support system on the motor housing module and is shown in a top view as a fold. The form of the sensor line portion 405 shown here is suitable for contacting the sensor line to the motor housing module and enables sensor integration on the sensor line portion 405. The form of the sensor line portion 405 substantially corresponds to that of the sensor line portion 405 described in FIG. 4, with the expansion of the sensor line portion 405 into a circumferential portion, which corresponds to the sensor groove, around the motor housing module. In this region of the expansion of the sensor line portion 405, three sensor carriers 410 are formed, by way of example, in the exemplary embodiment shown here. Electronic components, for example sensors, can be integrated on these sensor carriers 410. Here, the contact portion 415 additionally has an O-shaped recess 505, through which the feed-through line can be fed through if the contact portion 415 lies on the feed-through portion 205.

The contact portion 415 comprises at least one contact surface 510 for connecting to the at least one contact pin. The at least one contact surface 510 is formed in order to at least partially enclose the at least one contact pin. The contact surface 510 can also be referred to as a contact pad. According to the exemplary embodiment shown here, the contact portion 415 has, by way of example, four contact surfaces 510 in order to electrically connect four contact pins embedded in the feed-through portion to the sensor line portion 405. Depending on the form of the contact portion 415, the contact surfaces 510 can be semicircular or elliptical in order to at least partially enclose one contact pin each for electrically contacting with the sensor line portion 405. The forms of the motor housing module and of the sensor line are, for example, adapted to each other by the forming of the sensor line portion 405 such that the contact pads 510 enclose the contact pins of the motor housing module. For this purpose, the contact pads 510 have an exposed, electrically contactable region. An electrical contact can be established by solder or adhesive, for example. The contacting of the feed-through line to connect the motor can take place in the same way as the contacting of the contact pins with the sensors, or the contact portion 415 has, as shown here, the recess 505 in the shape of an O or U so that a connection of the feed-through line to the connection cable without contact to the contact portion 415 of the sensor line portion 405 is possible. The arrangement of the contact portion 415 on the feed-through portion, and thus the contacting of the sensor line to the blind pins of the motor housing module, can take place in the production process, e.g., by folding the sensor line portion 405 onto the feed-through portion and subsequently producing the electrical connections.

Additional installation space for accommodating electronic components, such as sensors, in the sensor groove can be created by additional depressions in the sensor groove of the motor housing module, in particular if the sensor line section 405 has several sensor carriers 410 as shown here. Components accommodated in the sensor groove can additionally be mechanically protected by a cap element.

According to one exemplary embodiment, an electronic component accommodated on the sensor line portion 405 in the sensor groove of the motor housing module can have a sensor hub. The sensor hub is designed to process at least one sensor signal of the at least one sensor of the heart support system. Additionally or alternatively, the sensor hub is designed to provide at least one sensor signal via the at least one contact pin to the connection cable. The integration of a sensor hub enables the pre-processing of sensor data and the translation of the data interfaces. In addition, calibration parameters and operating parameters, such as identification information of the heart support system or accommodated sensors, can be stored in the heart support system by means of the sensor hub and can be provided by means of the connection cable to a connected control device, e.g., via a communication bus in the connection cable. In this way, the control device can be parameterized with motor data, for example. The sensor hub can be used to pre-process, e.g., to aggregate, to filter, or to calibrate, sensor data from sensors of the heart support system pump and to translate the communication protocol of the sensors into a more robust communication protocol (transceiver) and add artificial redundancy or checksums.

FIG. 6 shows a schematic illustration of a cap element 605 for a motor housing module according to an exemplary embodiment. The cap element 605 is provided for use with the motor housing module of one of the figures shown here. The cap element 605 is formed to cover electronic components of a motor housing module, as described with reference to FIG. 3. The cap element 605 can therefore be used as mechanical protection of the motor housing module. A side view of the cap element 605 is shown in a one-piece design.

In the direction of the motor compartment, the cap element 605 has at least one recess 610 as a sensitive measurement window for one of the sensors 120/410/710. The sensor can, for example, be a pressure sensor so that the measurement window 610 is to be positioned above the pressure-sensitive membrane of the pressure sensor so that the blood pressure of the surrounding blood can act in an unimpeded manner on the pressure sensor. Adjacent to the recess 610, the cap element has the sensor cap 615. The sensor cap is formed in order to create a sensor groove, e.g., the sensor groove described in FIG. 3, which is formed by way of example as a depressed plane of the cylindrical body of the motor housing module. If the sensor groove is formed, for example, according to the exemplary embodiments described in FIGS. 4 and 5 as a circumferential depression, the sensor cap can be formed correspondingly to cover this region. Formed conically in the manner of an arrow tip, a connection point cap 620, which has an opening 625 for feeding through the connection cable, adjoins the sensor cap 615.

According to the exemplary embodiment shown here, the cap element 605 thus has the sensor cap 615 for covering the at least one electronic component accommodated in the sensor groove. The cap element 605 furthermore has the optional connection point cap 620 for covering a connection point between the feed-through portion and the connection cable. The sensor cap 615 and the connection point cap 620 can, as shown here, be designed as a combined one-piece component as cap element 605.

Alternatively, the sensor cap 615 and the connection point cap 620 can also be designed as respectively separate components. In this case, the sensor cap 615 can, for example, be a metallic cap that is fixed by gluing. The connection point cap 620 can be formed flexibly, for example, in order to enable bend protection and strain relief in addition to mechanical protection. The cap element 605 can, for example, be filled with a casting compound, e.g., a silicone or epoxy resin, in order to protect sensors and contact points from corrosion and conductive liquids.

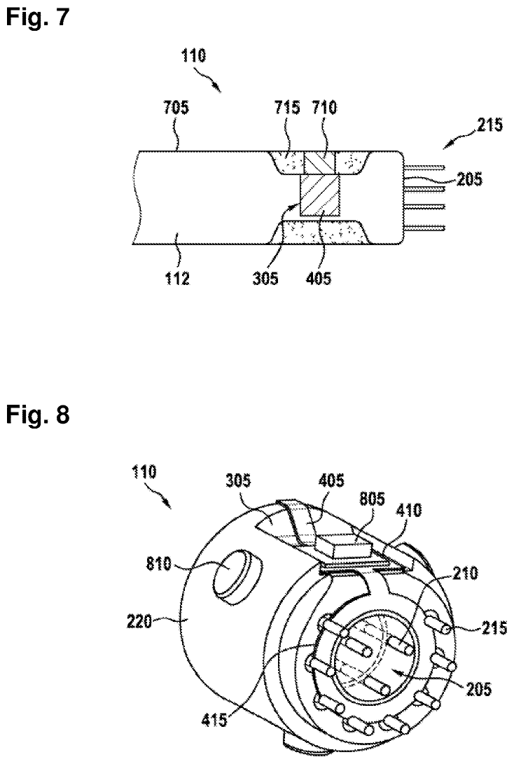

FIG. 7 shows a schematic illustration of a motor housing module 110 according to an exemplary embodiment. Here, the motor housing module 110 corresponds or resembles the motor housing module of one of the above-described figures. The side view shows, as a section of the mounted cylindrical heart support system, the motor compartment 112 with a motor compartment housing 705. The motor housing module 110 is connected to the motor compartment housing 705 and has, in the direction of the motor compartment 112, a circumferential depression as a sensor groove 305. In the region of the sensor groove 305, a sensor 710 is integrated, by way of example, as an electronic component on the sensor line portion 405. In order to illustrate the possibility of filling the sensor 710 with a casting compound using the sensor cap and/or the cap element, as described with reference to the previous FIG. 6, the correspondingly filled region 715 is shown here by way of example. On the side facing away from the motor compartment 112, the motor housing module 110 has the feed-through portion 205, from which four contact pins 215 project by way of example.

FIG. 8 shows a schematic illustration of a motor housing module 110 according to an exemplary embodiment. The motor housing module 110 is shown here in a top view. The body 220 is realized as a titanium part. For the electrical functionalization of the motor housing module 110 as an electrical connecting element, the sensor line portion 405 is laid from the direction of the motor compartment into the sensor groove 305. The sensor line portion 405 is here formed as a thin-film substrate by way of example. The body 220 in the form of a milled part made of titanium has a depressed plane as a sensor groove 305. The sensor line portion 405 expands in the region of the sensor groove 305 and, as a thin layer, almost completely fills a lower region of the base surface of the sensor groove 305. A sensor carrier 410, on which, by way of example, an electronic component 805 is accommodated, is located in the sensor groove 305 on the sensor line portion 405.

According to one exemplary embodiment, the motor housing module 110 has a coupling device for coupling with the motor housing module 110 an insertion device for inserting the heart support system, wherein the coupling device in particular has at least one fixing element 810. The fixing element 810 can serve for the form-fitting coupling of a clamp element, a so-called clamp. The body 220, as a titanium part, has here, by way of example, three round fixing elements 810 as a coupling device. The fixing elements 810 can additionally or alternatively also be used to fix a cap element for covering an electronic component 805 or an electrical connection point of the motor housing module 110; the fixing elements 810 then serve as a fit for attaching the cap element.

The exemplary embodiment of the motor housing module 110 shown here has the body 220 and the feed-through portion 205 realized as a so-called glass feed-through. By way of example, three feed-through lines 210 for electrically connecting the motor of the heart support system to the connection cable are embedded in the feed-through portion 205. In addition, eight contact pins 215 arranged in the shape of a U are embedded in the feed-through portion 205 by way of example. The contact pins 215 are spaced apart substantially evenly. Tapered in the shape of a band in the direction of the feed-through portion 205, the sensor line portion 405 is guided out of the sensor groove 305 and forms the O-shaped contact portion 415. Adjacent to the contact pins 215, the contact portion 415 respectively has a semicircular contact surface for electrically connecting the contact pins 215 to the sensor line portion 405. The connection cable can be connected to the feed-through line 210 and to the contact pins 215 in order to externally contact the heart support system by means of the motor housing module 110.

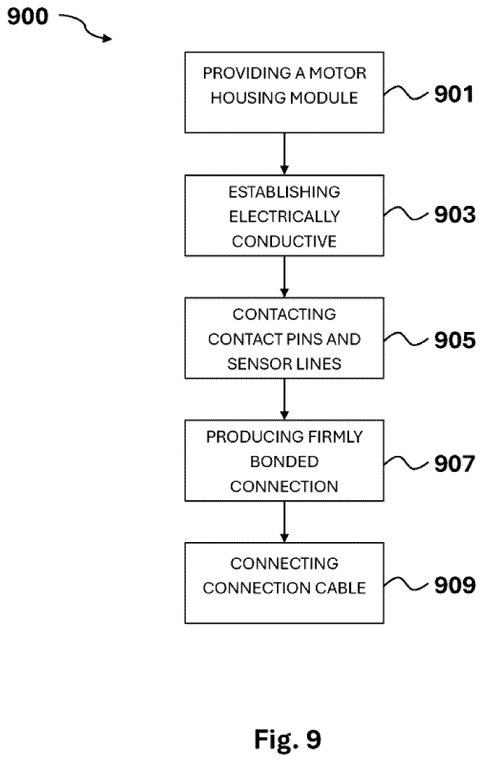

FIG. 9 shows a flow diagram of a method 900 for mounting a heart support system according to an exemplary embodiment. The heart support system has a motor, a motor compartment, at least one sensor, a sensor line electrically connected to the at least one sensor, and a connection cable for externally contacting the heart support system. The method 900 comprises a step 901 of providing, a step 903 of establishing, a step 905 of contacting, and a step 907 of producing. In step 901 of providing, a motor housing module is provided. Here, the motor housing module corresponds or resembles the motor housing module of one of the above-described figures. In step 903 of establishing, an electrically conductive connection is established between the at least one feed-through line of the motor housing module and the motor of the heart support system. In step 905 of contacting, the at least one contact pin of the motor housing module is contacted with the sensor line of the heart support system. In step 907 of producing, a firmly bonded connection is produced between the motor housing module and the heart support system in order to seal the motor compartment of the heart support system. In addition, in step 907 of producing, a sensor cap and/or a connection point cap for covering and protecting an electronic component or an electrically conductive interface of a component of the heart support system can optionally be mounted.

A sequence of the steps of the method presented here can also be provided in a special exemplary embodiment as follows:
1. Attaching the feed-through pin to the motor interior
2. Positioning the body 220
3. Tightly welding the body to the motor housing so that the connection established in this way is retained mechanically
4. Tightly welding the contact element in the body
5. Affixing the sensor line 125, folding the contact portion 415 onto the feed-through portion 205, contacting the contact surface 510 to the contact pin 215
6. Contacting the sleeves to wires of the connection cable 130
7. Sliding the contacted sleeves onto contact pin 215 and feed-through line 210 and welding them thereto
8. Casting and positioning the sensor cap 615 and the connection point cap 620

According to one exemplary embodiment, the method 900 optionally has a step 909 of connecting the connection cable of the heart support system to the at least one feed-through line and the at least one contact pin of the motor housing module. The step 909 of connecting can be carried out before or after step 907 of producing.

If an exemplary embodiment includes an “and/or” conjunction between a first feature and a second feature, this should be read to mean that the exemplary embodiment according to one embodiment comprises both the first feature and the second feature and according to another embodiment comprises either only the first feature or only the second feature.

1. (canceled)

2. A heart support system, comprising:
a blood pump;
a sensor disposed on a surface of the blood pump; and
a motor housing module comprising:
a body having a motor compartment therein, the motor compartment housing at least a portion of a motor of the blood pump;
a feed-through portion arranged outside the motor compartment and configured to establish an electrical connection between the heart support system and a connection cable;
at least one feed-through line extending through the feed-through portion, wherein the at least one feed-through line is configured to connect the motor to an exterior side of the motor housing module opposite the motor compartment; and
at least one contact terminal embedded in the feed-through portion on the exterior side of the motor housing module opposite the motor compartment, the at least one contact terminal configured to form a connection between the connection cable and the sensor via a connection external to the motor housing module.

3. The heart support system according to claim 2, wherein the at least one contact terminal is configured to connect to a conductor of the connection cable.

4. The heart support system according to claim 2, wherein the at least one contact terminal comprises at least one contact pin.

5. The heart support system according to claim 4, wherein the at least one feed-through line and/or the at least one contact terminal is cylindrical or cup-shaped.

6. The heart support system according to claim 2, wherein the motor housing module comprises a sensor groove configured to receive the sensor.

7. The heart support system according to claim 5, wherein the sensor groove is configured to receive the sensor, the heart support system further comprising a sensor cap positioned at least partially over the sensor groove and having a measurement window for the sensor within the sensor groove.

8. The heart support system according to claim 5, further comprising a sensor line portion comprising a sensor carrier in a region of the sensor groove, the sensor carrier being configured to connect to the sensor.

9. The heart support system according to claim 7, wherein the sensor groove is configured to receive a sensor hub, and wherein the sensor hub is configured to process at least one sensor signal of the sensor and/or to provide a sensor signal to the connection cable.

10. The heart support system according to claim 7, wherein the sensor line portion comprises a contact portion, wherein the contact portion is arranged on a side of the feed-through portion facing away from the motor compartment.

11. The heart support system according to claim 9, wherein the contact portion is O-shaped or U-shaped.

12. The heart support system according to claim 9, wherein the contact portion comprises at least one contact surface for connecting to the at least one contact terminal, and wherein the at least one contact surface is formed to at least partially enclose the at least one contact terminal.

13. The heart support system according to claim 2, having a connection point cap configured to cover a connection point between the feed-through portion and the connection cable.

14. The heart support system according to claim 2, further comprising a coupling device for coupling the motor housing module to an insertion device configured to insert the heart support system, wherein the coupling device comprises at least one fixing element.

15. The heart support system according to claim 2, wherein the heart support system is configured to be inserted into a heart chamber or an aorta by means of a catheter.

16. The heart support system according to claim 2, wherein the at least one contact terminal is configured to connect to the sensor via a sensor line.

17. A method for mounting a heart support system, wherein the heart support system comprises a blood pump, a sensor disposed on a surface of the blood pump, a sensor line electrically connected to the sensor, and a connection cable, wherein the method comprises:
providing a motor housing module, the motor housing module comprising:
a body having a motor compartment therein, the motor compartment housing at least a portion of a motor of the blood pump;
a feed-through portion configured to establish an electrical connection between the heart support system and the connection cable; and
at least one contact terminal embedded in the feed-through portion on an exterior side of the motor housing module opposite the motor compartment, and wherein the at least one contact terminal configured to form a connection between the connection cable and the sensor via a connection external to the motor housing module; and
contacting the at least one contact terminal of the motor housing module with the sensor line of the heart support system.

18. The method according to claim 16, further comprising connecting the connection cable of the heart support system to at least one contact pin of the motor housing module.

19. The method according to claim 16, wherein the at least one contact terminal is configured to connect to a conductor of the connection cable.

20. The method according to claim 16, wherein the at least one contact terminal comprises at least one contact pin.

21. The method according to claim 16, wherein the motor housing module comprises a sensor groove configured to receive the sensor.
